(19) United States
(12) Patent Application Publication
(10) Pub. No.: US 2018/0014971 A1
(43) Pub. Date: Jan. 18, 2018

(54) THERMALLY CONDUCTIVE GRAFT

(71) Applicants: University of Washington, Seattle, WA (US); Washington University in St. Louis, St. Louis, MO (US)

(72) Inventors: Raimondo D'Ambrosio, Seattle, WA (US); Samuel R. Browd, Seattle, WA (US); John W. Miller, Bellevue, WA (US); Jeffrey G. Ojemann, Seattle, WA (US); Clifford L. Eastman, Seattle, WA (US); Matthew Smyth, Frontenac, MO (US); Steven M. Rothman, Clayton, MO (US); Jason Fender, Bonney Lake, WA (US)

(21) Appl. No.: 15/714,518

(22) Filed: Sep. 25, 2017

Related U.S. Application Data
(63) Continuation of application No. PCT/US2016/024281, filed on Mar. 25, 2016.
(60) Provisional application No. 62/138,173, filed on Mar. 25, 2015.

(51) Int. Cl. A61F 7/12; A61F 7/00

(52) U.S. Cl. CPC A61F 7/12; A61F 2007/0002

(57) ABSTRACT

The present disclosure provides thermally conductive grafts and methods of passively cooling a hyperthermic region and preventing epilepsy, neural inflammation, and other neurological abnormalities using a thermally conductive graft including a thermally conductive matrix disposed between two opposed surfaces.

### CROSS-REFERENCE TO RELATED APPLICATIONS

This application is a continuation of International Application No. PCT/US2016/024281, filed Mar. 25, 2016, which claims the benefit of U.S. Provisional Application No. 62/138,173 filed Mar. 25, 2015, entitled “Central Nervous System Graft for the Treatment of Epilepsy and Neuroinflammation,” the contents of each are incorporated herein by reference in their entirety.

### BACKGROUND

Epilepsy can be understood as a syndrome involving episodic abnormal electrical activity in the brain, or epileptic seizures, that result from abnormal, excessive or hypersynchronous neuronal activity in the brain. It is estimated that 50 million people worldwide have epilepsy. The onset of epileptic symptoms occurs most frequently in infants and the elderly, and may also arise from trauma to the brain or as a consequence of brain surgery.

Epileptic symptoms are sometimes controllable with medication. However, nearly one-third (⅓) of persons with epilepsy cannot control seizures even with the best available medications. In certain cases, neurosurgery is undertaken to remove the epileptic focus to control the seizures.

For example, the high incidences of traumatic brain injury (TBI) in both the civilian and military populations, and the absence of any prophylactic treatment for acquired epilepsy, such as post-traumatic epilepsy (PTE), create an urgent need to develop broad-spectrum and easily deployable therapeutic strategies. There are currently no effective means for preventing the onset of PTE following head injury. The administration of anticonvulsants after head injury may decrease early post-traumatic seizures but has failed to impact the development of long-term epilepsy or improve the incidence of disability or death. Therefore, novel treatment paradigms are needed.

The process of epileptogenesis in humans is not known. It is theorized that agents that are neuro-protective may also be anti-epileptogenic. Similarly, the process of ictogenesis (i.e., the precipitation of seizures) is not necessarily the same as epileptogenesis. It is, therefore, entirely possible that treatments that prevent the precipitation of seizures do not prevent the genesis of epilepsy and, vice versa, those that may prevent the onset of epilepsy may not be capable of shutting down existing seizures.

There are known devices that use active cooling to shut down epileptic seizures (antiepileptic effect). Many known devices are based on the assumption that cooling a targeted area of the brain by about 1° C. is necessary to shut down the epileptic focus. One such device is based on active Peltier cells that cool the brain, including heat pipes to cool deep into the brain. A second known device uses circulating coolant in tubing implanted within the dorsal hippocampus of a brain to achieve cooling of at least 7° C. in the hippocampus. Unfortunately, such devices are typically highly intrusive (if inserted deep into the brain) and require the implantation of complex structures (e.g., heat pipes), electronics (e.g., Peltier elements), and long-lasting powering elements (e.g., batteries) to produce the necessary cooling.

Further, epileptic foci generate more heat than surrounding tissue. Several factors may affect the temperature of defined regions of brain parenchyma in general, and of an epileptic focus in particular.

Neuronal activity results in localized transient temperature increases. Suzuki et al. (2012) have recently used infrared thermography to image the activity-induced temperature increases in the rat barrel cortex in response to whisker stimulation, and shown the observed changes to be largely independent of changes in regional cerebral blood flow (rCBF). See Suzuki T, Ooi Y, Seki J., Infrared thermal imaging of rat somatosensory cortex with whisker stimulation. J. Appl. Physiol., 112(7):1215-22 (2012). Thus, the elevated neuronal activity and supporting metabolism in the epileptic focus may stably exceed that in adjacent tissue to give rise to a measurable temperature gradient both inter-ictally and ictally.

Inflammation generates heat. Micro-calorimetry studies have demonstrated that immune cells produce heat upon activation (Charlebois S J, Daniels A U, Smith R A., Metabolic heat production as a measure of macrophage response to particles from orthopedic implant materials, J. Biomed. Mater Res. January; 59(1):166-75 (2002); Hayatsu H, Masuda S, Miyamae T, Yamamura M., Heat production due to intracellular killing activity, Tokai J. Exp. Clin. Med. September; 15(5):395-9 (1990); Pärsson H, Nässberger L, Thörne J, Norgren L., Metabolic response of granulocytes and platelets to synthetic vascular grafts: preliminary results with an in vitro technique, J. Biomed. Mater Res. April; 29(4):519-25 (1995); Yamamura M, Hayatsu H, Miyamae T, Shimoyama Y., Heat production as a quantitative parameter for cell differentiation and cell function, Tokai J. Exp. Clin. Med., September; 15(5):377-80 (1990)). This heat generation may reflect activation-related increases in the rate of oxidative metabolism, the predominance of inefficient glycolytic metabolism in some immune cells (Geering B, Simon H U., Peculiarities of cell death mechanisms in neutrophils, Cell Death Differ. September; 18(9):1457-69 (1990)), or regulated uncoupling of mitochondrial respiration, which may play a role in phagocytosis (Cereghetti G M, Scorrano L. Phagocytosis: coupling of mitochondrial uncoupling and engulfment., Curr. Biol.; 21(20):R852-4 (2011); Park D, Han C Z, Elliott M R, Kinchen J M, Trampont P C, Das S, Collins S, Lysiak J J, Hoehn K L, Ravichandran K S., Continued clearance of apoptotic cells critically depends on the phagocyte Ucp2 protein, Nature.; 477(7363):220-4 (2011)). Recent evidence supports an important role for inflammation in epilepsy (Choi J, Koh S., Role of brain inflammation in epileptogenesis, Yonsei Med J.; 49(1):1-18 (2008); Fabene P F, Navarro Mora G, Martinello M, Rossi B, Merigo F, Ottoboni L, Bach S, Angiari S, Benati D, Chakir A, Zanetti L, Schio F, Osculati A, Marzola P, Nicolato E, Homeister J W, Xia L, Lowe J B, McEver R P, Osculati F, Sbarbati A, Butcher E C, Constantin G., A role for leukocyte-endothelial adhesion mechanisms in epilepsy, Nat. Med.; 14(12):1377-83 (2008); Friedman A, Dingledine R., Molecular cascades that mediate the influence of inflammation on epilepsy, Epilepsia., May; 52 Suppl 3:33-9 (2011); Li G, Bauer S, Nowak M, Norwood B, Tackenberg B, Rosenow F, Knake S, Oertel W H, Hamer H M, Cytokines and epilepsy, Seizure. April; 20(3):249-56 (2011)), and leukocyte infiltration has been observed in resected epileptic brain tissue from temporal lobe epilepsy patients (Zattoni M, Mura M L, Deprez F, Schwendener R A, Engelhardt B, Frei K, Fritschy J M., Brain infiltration of leukocytes contributes to the pathophysiology of temporal lobe epilepsy, J Neurosci.; 31(11):4037-50 (2011), and after status epilepticus, and even single brief seizures, in rodents (Fabene P F, Navarro Mora G, Martinello M, Rossi B, Merigo F, Ottoboni L, Bach S, Angiari S, Benati D, Chakir A, Zanetti L, Schio F, Osculati A, Marzola P, Nicolato E, Homeister J W, Xia L, Lowe J B, McEver R P, Osculati F, Sbarbati A, Butcher E C, Constantin G., A role for leukocyte-endothelial adhesion mechanisms in epilepsy, Nat. Med.; 14(12):1377-83 (2008); Kim et al., 2010; 2012; Silverberg et al., 2010; Zattoni et al., 2011).

Regulated mitochondrial uncoupling may contribute importantly to the elevated temperature of epileptic foci acquired after brain insult. When mitochondrial respiration is uncoupled from ATP production, the energy released from glucose oxidation is dissipated as heat. Three uncoupling proteins (UCP) are expressed in brain tissue at levels that may differ markedly between species (Alán L, SmolkováK, Kronusová E, Santorová J, Jezek P., Absolute levels of transcripts for mitochondrial uncoupling proteins UCP2, UCP3, UCP4, and UCP5 show different patterns in rat and mice tissues, J. Bioenerg. Biomembr.; 41(1):71-8 (2009)). UCP2 mRNA is ubiquitously expressed in all tissues but is strongly associated with immune cells (Alan et al., 2009). In the brain, UCP2 protein is expressed mainly in microglia (Rupprecht A, Bräuer A U, Smorodchenko A, Goyn J, Hilse K E, Shabalina I G, Infante-Duarte C, Pohl E E., Quantification of uncoupling protein 2 reveals its main expression in immune cells and selective up-regulation during T-cell proliferation, PLoS. One.; 7(8):e41406. doi: 10.1371/journal.pone.0041406. (2012)). Other UCP are induced by a variety of brain injuries, including ischemia-reprofusion, kainic acid and embolic stroke.

What is desired, therefore, is an improved device for preventing and/or treating acquired epilepsy and other neurological abnormalities.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Epilepsy can be mitigated or prevented by a method comprising removing a portion of a cranium through an incision in a scalp of a patient to form a recess in which a portion of dura mater is exposed; and implanting a cooling device adjacent the exposed portion of dura mater and closing the incision such that an adjacent portion of the brain is cooled by the cooling device by heat dissipation through the scalp, and wherein the adjacent portion of the brain is cooled by not more than 4° C., wherein the cooling device comprises a passive cooling device having a highly thermally conductive portion adjacent the exposed portion of dura mater. See, for example, U.S. patent application Ser. No. 13/482,903 published as U.S. 2012/0290052 and U.S. Pat. No. 8,591,562, each of which is hereby incorporated by reference in their entirety.

In certain instances, it may be beneficial to have the skull of the patient intact covering, for example, the epileptic focus or portion of the brain under which the passive cooling device resides. For example, a patient may be in an area where the ambient temperature is above the patient's body temperature. In such an instance, the transcranial device would direct heat from the surrounding environment to the brain, which is believed to be the opposite of the preferred direction.

Accordingly, in one aspect the present disclosure provides a thermally conductive graft comprising: a thermally conductive matrix, wherein the thermally conductive graft comprises a first surface, a second surface, and a thermally conductive matrix disposed between the first and second surfaces.

In a second aspect the present disclosure provides a method of passively cooling a hyperthermic region of the central nervous system comprising: implanting a thermally conductive graft adjacent to the hyperthermic region of the central nervous system, wherein the thermally conductive graft is effective to conduct heat from the hyperthermic region to another region.

In a third aspect the present disclosure provides a method of preventing or treating a neurological abnormality comprising: implanting a thermally conductive graft adjacent to a hyperthermic region of the central nervous system, wherein the thermally conductive graft conducts heat from the hyperthermic region to a region of the central nervous system that is not hyperthermic.

In a fourth aspect the present disclosure provides a method of preventing or treating inflammation of the central nervous system comprising: implanting a thermally conductive graft adjacent to a hyperthermic region of the central nervous system, wherein the thermally conductive graft conducts heat from the hyperthermic region to a region of the central nervous system that is not hyperthermic.

In a fifth aspect the present disclosure provides a method of preventing or treating a neurological abnormality comprising implanting a thermally conductive graft adjacent to a hyperthermic region of a central nervous system of a subject. In various examples, the thermally conductive graft comprises a first surface, a second surface, and a thermally conductive matrix disposed between the first and second surfaces

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts representations of infrared thermal images of rats with fluid-percussion injury.

FIG. 2A depicts a representation of an infrared image at the time of cortical resection for right frontal intractable epilepsy.

FIG. 2B depicts independent determination of the seizure focus from the view point shown in FIG. 2A.

FIG. 2C depicts pre-resection from the view point shown in FIG. 2A.

FIG. 3 illustrates relative expression of genes encoding pro-inflammatory cytokines after head injury in the rat.

FIG. 4A depicts a central nervous system graft according to an example embodiment of the present disclosure.

FIG. 4B depicts a central nervous system graft wherein the central nervous system graft is extended through an opening in the skull to a subgaleal space, in accordance with an example embodiment of the present disclosure.

FIG. 4C depicts a central nervous system graft replacing a portion of removed dura, in accordance with an example embodiment of the present disclosure.

FIG. 4D depicts the central nervous system graft of FIG. 4C, and further includes an illustration of passive heat dissipation from hyperthermic regions of the brain, in accordance with an example embodiment of the present disclosure.

### DETAILED DESCRIPTION

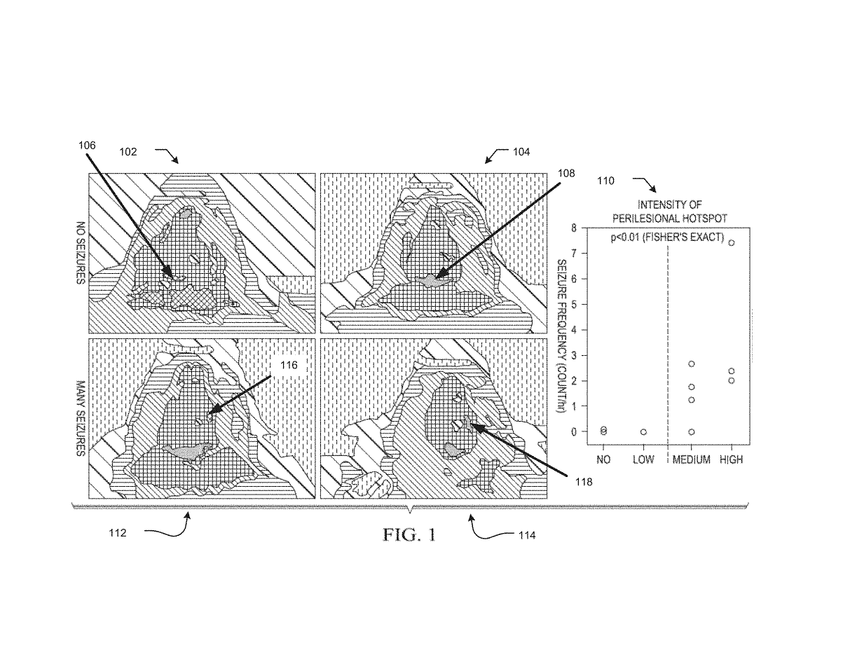

FIG. 1 depicts representations of infrared thermal imaging of rats with fluid-percussion injury. In the top panels 102 and 104, representations of thermal images of ‘no seizure’ rat brains are depicted. In top panels 102 and 104, only large veins in the posterior aspect of the cranial window at sites 106 and 108 show increased temperature (0.38±0.1° C. from hottest spot to rest of the cortex). In the bottom panels 112 and 114, two animals with many seizures show increased temperature (1.0±0.1° C.) along the edge of the neocortical injury at sites 116 and 118, relative to surrounding cooler areas of the cortex. The graph 110 depicts the relationship between seizure frequency and temperature intensity.

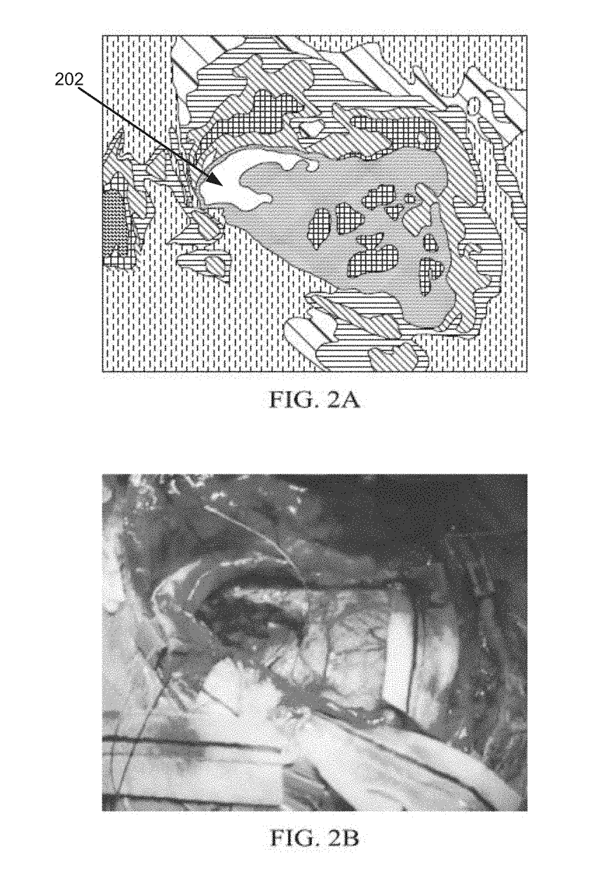

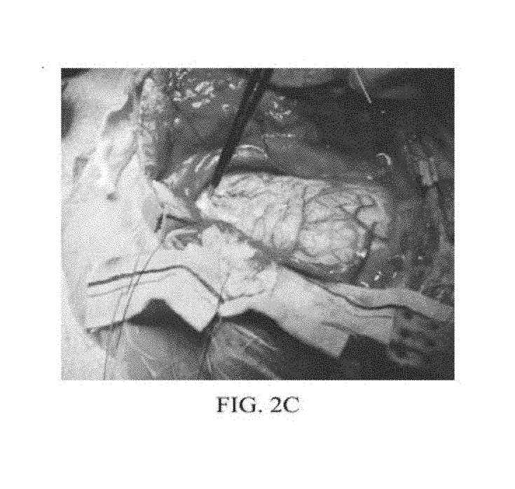

The presence of neocortical, hyperthermic “hot spots” in head-injured rats (shown in FIG. 1) and in drug resistant epilepsy patients (shown in FIGS. 2A-2C) may be observed using infrared thermal imaging. The hyperthermic “hot spots” may coincide with the positions of epileptic foci identified by electrocorticography (“ECoG”) in head-injured rats. FIG. 2A depicts a representation of an infrared image of a human epilepsy patient at the time of cortical resection for right frontal intractable epilepsy. The representation of the thermal image in FIG. 2A shows a higher temperature ‘hot spot’ 202 in the frontal region (i.e., the left side of the exposure) relative to the surrounding cortex. For example, the patient depicted in FIG. 2A experienced a peak temperature of 39.3° C. at hot spot 202 in the frontal region, while the entire exposed cortex including the hot spot 202 experienced a temperature of 37.1° C. FIG. 2B depicts independent determination of the seizure focus, with the superior frontal region being the ictal onset zone with resection of this area evident in the post-operative photograph. FIG. 2C depicts pre-resection from the same viewpoint as FIG. 2A.

In both head injured epileptic rats and humans, hot spots are evident during anesthesia, which fully controls epileptic activity (Eastman et al., 2010). Thus, the hot spots are not directly attributable to seizures or seizure-induced changes in regional cerebral blood flow, but to a more chronic process such as inflammation.

Regions of increased temperature that overlap with ictal onset zones (e.g., hot spot 202 of FIG. 2A) have been observed in human epilepsy patients who were studied, under anesthesia, during implantation of grids used for diagnosis and localization of their epileptic foci. The ictal onset zones were warmer than surrounding tissue by 2° C. or more.

These observations demonstrate that epileptic foci in both animals and humans are hyperthermic, i.e. at a higher temperature than the normal brain.

Mild-passive cooling with trans-cranial devices can be used to treat epilepsy. See, e.g., U.S. patent application Ser. No. 13/482,903 and U.S. Pat. No. 8,591,562, each of which is incorporated herein by reference in its entirety.

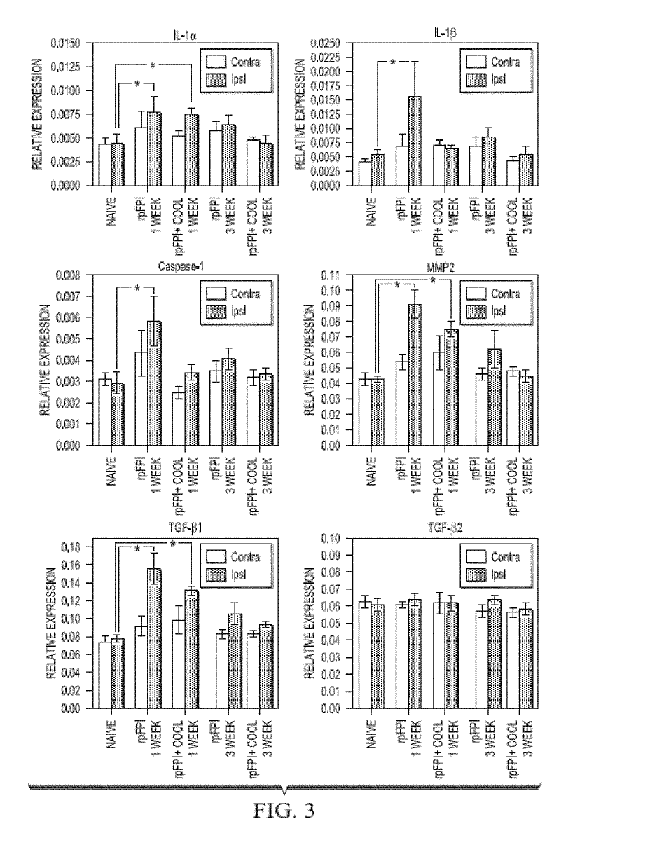

FIG. 3 illustrates relative expression of genes encoding pro-inflammatory cytokines after head injury in the rat. Mild passive focal cooling in the rat brain may be associated with anti-inflammatory effects. In the example depicted in FIG. 3, expression was measured in the contra- and ipsilateral neocortices of head injured rats randomized to cooling or no treatment, and in naive controls (n=7 animals per group), 1 week after injury (after 4 days of cooling). Expression was normalized to the geometric mean of 3 housekeeping genes with statistical comparison at p<0.05 vs. control.

Epileptic foci are inflamed and mild focal cooling is anti-inflammatory. In FIG. 3, anti-inflammatory effect on the inflamed epileptic focus is realized using mild focal cooling by 2° C. RT-PCR was used to examine the gene expression of pro- and anti-inflammatory cytokines after injury (4 days after cooling) before the appearance of focal seizures. Pro-inflammatory cytokines known to be involved in post-traumatic sequelae and epileptogenesis were elevated by FPI, and decreased by mild cooling. In particular, mild cooling had a dramatic effect on IL-1β nd caspase-1, both implicated in epileptogenesis. TGF-2(3 expression, which has not been implicated in epileptogenesis or TBI, was not affected by head injury or by cooling.

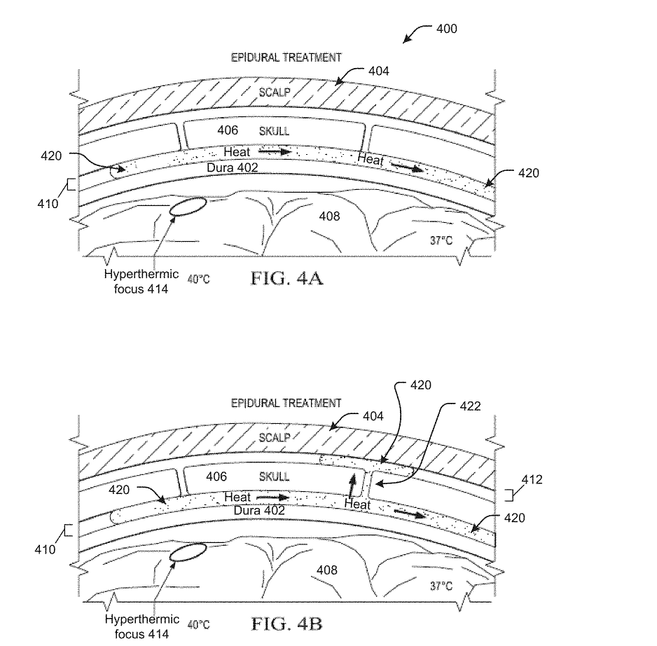

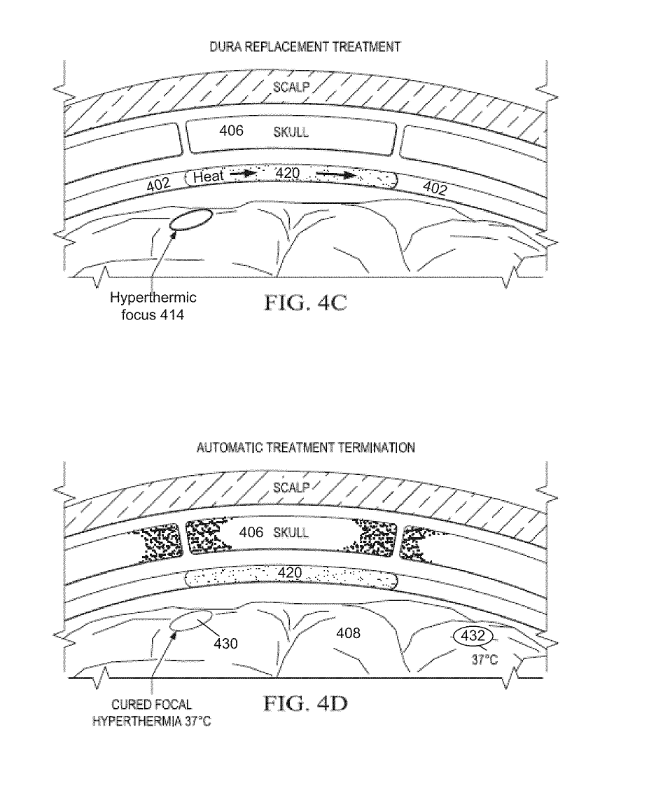

FIG. 4A depicts a system 400 including a thermally conductive graft 420 according to an example embodiment of the present disclosure. Thermally conductive graft 420 may be sized and configured to overlay the dura 402. Dura 402 may be a thick membrane that is the outermost of the three layers of the meninges that surround the brain 408 and spinal cord. In various examples, thermally conductive graft 420 may be sized, shaped and configured to fit in epidural space 410 between dura 402 and the skull of the patient.

A portion of skull 406 may be removed in a craniotomy to allow for placement of thermally conductive graft 420 between the skull and dura 402 in epidural space 410. In various examples, once thermally conductive graft 420 is placed overlaying dura 402, replacement of portion of skull 406 may compress thermally conductive graft 420. Such compression may effectively hold thermally conductive graft 420 in the desired position relative to the hyperthermic focus 414. As shown in FIG. 4A, thermally conductive graft 420 may be positioned so as to extend laterally beyond the edges of the craniotomy such that thermally conductive graft 420 underlies the entirety of portion of skull 406, and extends beyond the edges of the incision in the skull made during the craniotomy. In some other examples, thermally conductive graft 420 may underlie less than the entirety of portion of skull 406 removed during the craniotomy.

For the thermally conductive grafts depicted in FIGS. 4A-D, where the patient has a single epileptic and/or hyperthermic focus 414 in the patient's brain, thermally conductive graft 420 may be sized, positioned, and/or configured to overlay all, or substantially all of epileptic and/or hyperthermic focus 414. In some examples, thermally conductive graft 420, as depicted in FIGS. 4A-D, may be sized, positioned, and/or configured to overlay a portion of epileptic and/or hyperthermic focus 414. For example, thermally conductive graft 420 may be positioned to overlay ˜10-90% of epileptic and/or hyperthermic focus 414. In some other examples where the patient has multiple epileptic and/or hyperthermic foci 414, thermally conductive graft 420 may be sized, positioned, and/or configured to overlay all of the epileptic and/or hyperthermic foci 414 or a subset of all of the epileptic and/or hyperthermic foci 414.

In various examples, thermally conductive graft 420 may include a thermally conductive matrix. In some examples, the thermally conductive matrix may include a biocompatible matrix and a thermally conductive material embedded into the biocompatible matrix. A biocompatible material may be, for example, a material that is suitable for contact with bodily tissues and fluids because it does not cause an allergic reaction, immune response, or other significant adverse side effects. A matrix may be, for example, a three dimensional structure or scaffolding which may comprise repetitive polymeric elements at a molecular level. In various examples, the biocompatible matrix may include silicon, collagen, carbohydrate chains, expanded polytetrafluoroethylene, polylactides, polyglycolides, gelatin, agar, cellulose-based compounds, thermally conductive polymers, pericranium harvested from the patient, fascia lata, tissue harvested via autograph, allograph, and/or xenograph, and combinations thereof. In various examples, the thermally conductive material may include thermally conductive polymers, graphene, carbon nanotubes, diamond, metal powders, metal beads, and combinations thereof.

In various examples, thermally conductive graft 420 may be formed in such a way as to include one or more apertures extending from one substantially planar surface of thermally conductive graft 420 to another substantially planar surface of thermally conductive graft 420. Such an aperture (not shown) may allow fluid to drain between the substantially planar surfaces of thermally conductive graft 420 (e.g., from epidural space 410 to subgaleal space 412). In various other examples, thermally conductive graft 420 may include an aperture configured to allow fluid to drain laterally from one portion of thermally conductive graft 420 to the other. For example, thermally conductive graft 420 may be formed in such a way as to include an aperture (not shown) which extends in a direction that is parallel to the substantially planar opposed surfaces of thermally conductive graft 420. Such an aperture may allow fluid to drain laterally, from one portion of the thermally conductive graft to another (e.g., from the left hemisphere of the brain to the right hemisphere).

In some examples, the thermally conductive material may be dispersed throughout the biocompatible matrix of thermally conductive graft 420 in a uniform or semi-uniform manner. In an example, graphene may be the thermally conductive material. In the example, graphene powder may be diluted in a mixture of alcohol and water to form a solution. The water may be allowed to evaporate. Silicone may be mixed with the solution. The graphene may be evenly distributed into the silicone through mixing or homogenization prior to the silicone curing. The silicon may then cure and the alcohol may evaporate. In the example, silicon may comprise the biocompatible matrix with graphene comprising a thermally conductive material dispersed throughout the biocompatible matrix. In some examples, thermally conductive graft 420 may comprise a liquid or aerosol which forms into a solid or semi-solid biocompatible matrix, through, for example, exposure to an activator agent or exposure to the atmosphere.

In various other examples, thermally conductive graft 420 may comprise a thermally conductive metal sheet including biocompatible material such as titanium. In examples where thermally conductive graft 420 comprises a metal sheet, the thermally conductive graft 420 may be made to conform to the contours of the skull, dura, and/or brain of the particular patient into whom the thermally conductive graft 420 is to be implanted. For example, a thermally conductive titanium sheet may be 3D printed based on the curvature of a portion of the patient's skull which may have one or more underlying hyperthermic foci. In various examples, a thermally conductive metal sheet may be formed with a thickness of between 1 and 4 millimeters. In examples where thermally conductive graft 420 comprises a metal sheet, portion of skull 406 may be filed, shaved, or otherwise reduced according to the thickness of the metal sheet to allow space for thermally conductive graft 420 between the skull and the meningeal layer.

Thermally conductive graft 420 may be effective to conduct heat from a hyperthermic region to another region as shown by arrows in FIG. 4A. For example, thermally conductive graft 420 may be effective to conduct heat from a hyperthermic region on or in the patient's brain to a surrounding, cooler area of the patient's cortex. Thermally conductive graft 420 may at least partially overlay hyperthermic focus 414, which may be, for example, an epileptic focus, inflammation site, or other area of the brain with an elevated temperature relative to surrounding tissue. For example, hyperthermic focus 414 may have a temperature that is higher than 37° C. In another example, hyperthermic focus 414 may have a temperature that is higher than an average temperature of brain 408. Thermally conductive graft 420 may be comprised of a suitable material effective to passively conduct heat from the hotter epileptic focus or inflammation site to cooler surrounding areas of the patient's cortex.

In various examples, thermally conductive graft 420 may include substantially planar opposed surfaces. For example, a first substantially planar surface of thermally conductive graft 420 may be disposed adjacent to the skull of the patient while a second substantially planar surface of thermally conductive graft 420 may be disposed adjacent to a meningeal membrane of the patient, such as, for example, dura 402. In another example, a substantially planar surface of thermally conductive graft 420 may be disposed adjacent to the patient's brain. Although surfaces of thermally conductive graft 420 are described in various examples herein as including substantially planar opposed surfaces, such surfaces may curve to conform to the contours of the patient's skull, meningeal membrane, and/or brain, as appropriate.

In various examples, one or more of the substantially planar opposed surfaces may include a coating. For example, a planar surface of thermally conductive graft 420 may be coated with an adhesive material or may be formed in such a way as to adhere to the meninges of a patient. In some examples, a planar opposed surface of thermally conductive graft 420 may include grips, teeth, protrusions, and/or a sticky or tactile surface effective to prevent the thermally conductive graft 420 from sliding or becoming dislodged after being positioned by a surgeon. In some other examples, one or more of the substantially planar opposed surfaces of thermally conductive graft 420 may include a coating or surface that is non-scarring to the meninges of the patient. In various other examples, a surface of thermally conductive graft 420 may include a medicament or non-fouling coating. Non-fouling coatings may include, for example, polyethylene glycol (PEG) and/or zwitterionic polymers. In various examples, the medicament or non-fouling coating may aid in the prevention of infection and/or may have an anti-inflammatory effect.

FIG. 4B depicts thermally conductive graft 420, wherein the thermally conductive graft 420 is extended through an opening in the skull to a subgaleal space 412, in accordance with an example embodiment of the present disclosure. Subgaleal space 412 may be, for example, an area between the skull and the scalp. As depicted in FIG. 4B, thermally conductive graft 420 may be disposed between dura 402 and the skull and may be disposed in a channel 422 which extends through the skull from epidural space 410 to subgaleal space 412. Channel 422 may be, for example, a burr hole, aperture, or other incision in the skull. For example, channel 422 may be an incision formed during a craniotomy. As depicted in FIG. 4B, thermally conductive graft 420 may extend from epidural space 410 through channel 422 and laterally in one or more directions from channel 422 in subgaleal space 412. Such a configuration may allow heat to dissipate from hyperthermic focus 414 to cooler regions of the brain and also to the scalp through channel 422.

In some examples, channel 422 and thermally conductive graft 420 may be used in conjunction with an active heat pump to transfer heat to or from the brain. For example, a Peltier device or other heat pump may be coupled to the patient's scalp, the portion of thermally conductive graft 420 in subgaleal space 412, or directly to the portion of the thermally conductive graft 420 residing in channel 422. The Peltier device or other heat pump may be activated to accelerate the flow of heat from hyperthermic focus 414 through thermally conductive graft 420 (including the portion of thermally conductive graft 420 residing in channel 422) to the environment outside the patient's head.

FIG. 4C depicts thermally conductive graft 420 replacing a portion of removed dura 402, in accordance with an example embodiment of the present disclosure. In such embodiments, the surgeon may remove a portion of native dura 402 and/or other meningeal layers and replace the removed portion with thermally conductive graft 420 rather than overlaying thermally conductive graft 420 on top of dura 402. For example, the patient's native dura may become damaged as a result of head trauma. The damaged native dura may be replaced with thermally conductive graft 420. In examples where thermally conductive graft 420 replaces native dura 402, thermally conductive graft may be of approximately the same thickness, or slightly thicker than, native dura 402. Heat dissipation properties of thermally conductive graft 420 may have beneficial anti-inflammatory effects at the site of the traumatic brain injury.

In some examples, thermally conductive graft 420 may comprise a suturable material, such as a suturable collagen, and may be sutured to the surrounding dura 402 and/or to the other surrounding meningeal layers. In various examples, suturing thermally conductive graft 420 may prevent leakage of spinal fluid. In some other examples, thermally conductive graft 420 may be a non-suturable biocompatible matrix and may be compressed into a “well” or “divot” left by the removal of a portion of dura 402 and/or other meningeal layers. Compression of non-suturable thermally conductive graft 420 may be caused by replacement of portion of skull 406 which was removed during a craniotomy. Compression of thermally conductive graft 420 may prevent leakage of spinal fluid between the remaining native dura 402 and thermally conductive graft 420. In some examples, replacing a portion of dura 402 with thermally conductive graft 420 may allow for efficient dissipation of heat from hyperthermic focus 414 to cooler portions of the brain. For example, heat may be conducted through thermally conductive graft 420 to cooler portions of the brain relative to hyperthermic focus 414, as shown by arrows in FIG. 4C. Additionally, the removed portion of dura 402 is no longer able to act as a heat-insulating layer on top of the brain which may further increase the efficiency of heat transfer away from hyperthermic focus 414.

In various embodiments, one or more heat pipes may be thermally coupled to thermally conductive graft 420. For example, a heat pipe may be positioned within the brain of the patient and may be effective to transfer heat away from a hyperthermic focus 414 which lies below the surface of the brain. A first end of a heat pipe may extend into the brain to an area which is proximate to the hyperthermic focus. A second end of the heat pipe may be embedded in, or otherwise coupled to, thermally conductive graft 420. In such an example, heat may be transferred from the first end of the heat pipe to the second end of the heat pipe and into the thermally conductive matrix. Heat may then be transferred through the thermally conductive matrix to cooler portions of the brain and/or to the scalp, according to various implementations of thermally conductive graft 420 described herein.

FIG. 4D depicts the thermally conductive graft 420 of FIG. 4C, and further includes an illustration of passive heat dissipation from hyperthermic regions of the brain, in accordance with an example embodiment of the present disclosure. As shown in FIG. 4D, replacing a portion of dura 402 with thermally conductive graft 420 may allow heat to dissipate from a hyperthermic focus to cooler areas of the brain, lowering the temperature of the focus until it is no longer hyperthermic. In an example depicted in FIG. 4D, the cured focal hyperthermia region 430 is shown to have a temperature of 37° C. which is the same as the temperature at a distal region 432 of brain 408. When a focus is no longer hyperthermic, the temperature gradient breaks down and the thermally conductive matrix of thermally conductive graft 420 ceases to transfer heat. Advantageously, if another hyperthermic focus arises underlying thermally conductive graft 420 at a later time, thermally conductive graft 420 will resume passive heat transfer to cooler areas of the brain without requiring any external input or activation.

Among other potential benefits, a thermally conductive graft 420 arranged in accordance with various embodiments described herein may be used to treat or prevent seizures. Additionally, in some embodiments, a thermally conductive graft 420 may be used to reduce inflammation by cooling inflamed areas, such as a site of traumatic injury. Reduction of inflammation may in turn reduce scarring which may be beneficial particularly in the context of follow-up procedures where native and/or non-native materials may fuse together via scar tissue. Additionally, although described herein primarily in the context of brain surgery, thermally conductive grafts may also be used in different contexts to focally cool hyperthermic areas of tissue. For example, thermally conductive grafts as described herein may be used to focally cool an inflamed area following removal of a spinal cord tumor or following other surgery. Furthermore, the thermally conductive graft may continue to automatically function to transfer heat in case of a reoccurrence of a hyperthermic region or a newly arisen hyperthermic focus.

In accordance with the above discovery, in one aspect, the present disclosure provides a thermally conductive graft 420 comprising a thermally conductive matrix, wherein the central nervous system graft has substantially planar opposed surfaces and is sized and configured to fit between the brain and the skull.

As used herein, the “central nervous system” is the part of the nervous system that integrates information it receives from, and coordinates and influences the activity of, all parts of the body of a bilaterally symmetric animal. It includes the brain, spinal cord, and proximal ganglia.

In certain embodiments, thermally conductive graft 420 is sized and configured to replace a meningeal membrane. In certain other embodiments, thermally conductive graft 420 is sized and configured to overlay a native meningeal membrane.

In certain embodiments, thermally conductive graft 420 further comprises at least one thermally conductive subcutaneous strip that extends away from the graft surface and is configured to be positioned adjacent to the meninges. In certain further embodiments, the thermally conductive graft 420 is sized and configured to extend beyond the edge of a craniotomy through to a subgaleal space.

In a second aspect, the present disclosure provides a method of passively cooling a hyperthermic region of the central nervous system comprising implanting a thermally conductive graft adjacent to a hyperthermic region of the central nervous system, wherein the thermally conductive graft conducts heat from the hyperthermic region to another region.

As used herein, a “hyperthermic region” of a brain is an area of the brain that has an abnormally high temperature. In certain embodiments, the hyperthermic region has temperature above 37° C. prior to treatment. In certain embodiments, the hyperthermic region of the brain has a temperature that is higher than the average temperature of the brain.

In certain embodiments of the present disclosure, the hyperthermic region is an epileptic focus.

As used herein, an “epileptic focus” is the location of the epileptic abnormality or area from which seizures may develop.

In certain embodiments, the method further comprises removing a portion of the dura mater adjacent to the hyperthermic region.

In certain embodiments, the method further comprises replacing the portion of the cranium adjacent to the hyperthermic region.

In certain embodiments, the thermally conductive central nervous system graft is sized and shaped to substantially overlay the hyperthermic region and extend away from the hyperthermic region between the brain and the skull.

In a third aspect, the present disclosure provides a method of preventing or treating a neurological abnormality comprising: implanting a thermally conductive graft adjacent to a hyperthermic region of the central nervous system, wherein the thermally conductive graft conducts heat from the hyperthermic region to a region of the central nervous system that is not hyperthermic.

In certain embodiments, the neurological abnormality is selected from a group consisting of epilepsy, stroke, and traumatic brain injury.

In certain embodiments, the neurological abnormality is epilepsy. In various embodiments, the pathological effect or symptom of epilepsy may comprise at least one of convulsive seizures, focal seizures, and generalized seizures (including tonic-clonic, tonic, clonic, myoclonic, absence, and atonic seizures), and a post-ictal state of confusion.

In certain embodiments, the method further comprises removing a portion of the dura mater adjacent to the hyperthermic region.

In certain embodiments, the method further comprises replacing the portion of the cranium adjacent to the hyperthermic region.

In certain embodiments, the thermally conductive central nervous system graft is sized and shaped to substantially overlay the hyperthermic region and extend away from the hyperthermic region between the brain and the skull.

In certain other embodiments, the central nervous system graft is sized and configured to partially cover the hyperthermic region.

In certain other embodiments, the central nervous system graft is sized and configured to be adjacent to the hyperthermic region. In certain further embodiments, the central nervous system graft is within 0.1 mm, 0.2 mm, 0.3 mm, 0.4 mm. 0.5 mm, 0.6 mm, 0.7 mm, 0.8 mm, 0.9 mm, 1.0 mm, or more away from the hyperthermic region.

In a fourth aspect the present application provides a method of preventing or treating inflammation of the central nervous system comprising: implanting a thermally conductive graft adjacent to a hyperthermic region of the central nervous system, wherein the thermally conductive graft conducts heat from the hyperthermic region to a region of the central nervous system that is not hyperthermic.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

Advantages of the devices and methods according to the present application.

First, a thermally conductive graft may require simple materials to build. The portion of the skull removed during surgery can be merely replaced. The technology to create gel or silicone pads already exists. Similarly, collagen based autograph, allograph, and zenograph dural replacements exist in various forms and can be augmented in accordance with the present technology.

Second, the cooling action is not significantly affected by the temperature of the scalp. This might be an issue when the epilepsy patient remains in a particularly cold environment for a protracted period of time. The scalp could cool below body temperature, thus further cooling the underlying portion of the brain.

Third, the amount of treatment is directly related to the pathology to be addressed (e.g. focal hyperthermia). For example, the warmer the inflamed region of the central nervous system, the greater the cooling effect. If the central nervous system tissue dis-inflames over time and the temperature normalizes, the temperature gradient collapses, and the thermally conductive graft will automatically terminate the cooling effect. The thermally conductive graft may also resume passive cooling in case of recrudescence of the pathological hyperthermia.

Fourth, the thermally conductive graft can be conveniently used in a variety of neurosurgical applications, where acute inflammation complicates outcome. By replacing a portion of the dura and cooling the underlying brain tissue, the thermally conductive graft may produce an anti-inflammatory treatment that may prove beneficial for a wide range of brain injuries or neurological disorders, and also to abate acute inflammation after any neurosurgical treatment of a portion of the central nervous system. Such passive cooling may improve the outcome of almost any neurosurgical treatment.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

What is claimed is:

1. A thermally conductive graft comprising:
a first surface;
a second surface; and
a thermally conductive matrix disposed between the first and second surfaces.

2. The thermally conductive graft of claim 1, wherein the thermally conductive matrix comprises: a biocompatible matrix and a thermally conductive material embedded into the biocompatible matrix.

3. The thermally conductive graft of claim 2, wherein the thermally conductive material comprises one or more of the following: thermally conductive polymers, graphene, carbon nanotubes, diamond, metal powders, metal beads, and combinations thereof.

4. The thermally conductive graft of claim 2, wherein the biocompatible matrix comprises one or more of the following: silicon, collagen, expanded polytetrafluoroethylene, polylactide, polyglycolide, gelatin, agar, cellulose, thermally conductive polymer, carbohydrate chain, collagen autograph, allograph, xenograph, and combinations thereof.

5. The thermally conductive graft of claim 1, wherein the thermally conductive graft is sized and configured to: (1) overlay a meningeal membrane under a skull of a human subject; or (2) replace a portion of the meningeal membrane of the human subject.

6. The thermally conductive graft of claim 1, wherein the thermally conductive graft is sized and configured to extend from a dural space through a channel in a skull to a subgaleal space of a human subject.

7. The thermally conductive graft of claim 1, wherein the thermally conductive matrix comprises a biocompatible polymer matrix.

8. The thermally conductive graft of claim 1, wherein the thermally conductive graft is between 0.1 mm and 8 mm thick.

9. The thermally conductive graft of claim 1, wherein the second surface comprises a coating which is adhesive to a meninges of a human subject.

10. The thermally conductive graft of claim 1, wherein the second surface comprises a coating which is non-scarring to a meninges of a human subject.

11. The thermally conductive graft of claim 1, further comprising a non-fouling coating on one or more of the first surface and the second surface.

12. The thermally conductive graft of claim 1, further comprising at least one aperture disposed in the thermally conductive matrix sized and configured to allow fluid to drain from one substantially planar opposed surface to the other.

13. The thermally conductive graft of claim 1, further comprising at least one aperture disposed in the thermally conductive matrix having a first and a second end sized and configured to allow fluid to drain laterally from portion of the thermally conductive graft to the other.

14. The thermally conductive graft of claim 1, wherein the first surface is substantially planar and the second surface is substantially planar.

15. A method of passively cooling a hyperthermic region of a central nervous system of a human subject, the method comprising:
implanting a thermally conductive graft adjacent to the hyperthermic region of the central nervous system,
wherein the thermally conductive graft is effective to conduct heat from the hyperthermic region to another region.

16. The method of claim 15, further comprising:
making an incision in a scalp of a subject;
removing a portion of a cranium through the incision to form a recess in which a portion of a meningeal membrane adjacent to the hyperthermic region is exposed; and
implanting the thermally conductive graft adjacent to the exposed meningeal membrane.

17. The method of claim 15, wherein the thermally conductive graft comprises:
a first surface;
a second surface; and
a thermally conductive matrix disposed between the first and second surfaces.

18. The method of claim 17, wherein the thermally conductive matrix comprises a biocompatible matrix and a thermally conductive material embedded in the biocompatible matrix.

19. The method of claim 18, wherein:
the thermally conductive matrix comprises one or more of the following: thermally conductive polymers, graphene, carbon nanotubes, diamond, metal powders, metal beads, and combinations thereof; and
the biocompatible matrix comprises one or more of the following: silicon, collagen, expanded polytetrafluoroethylene, polylactide, polyglycolide, gelatin, agar, cellulose, thermally conductive polymer, carbohydrate chain, collagen autograph, allograph, xenograph, and combinations thereof.

20. The method of claim 15, further comprising: (1) removing a portion of a dura mater adjacent to the hyperthermic region of the human subject; or (2) replacing a portion of a cranium of the human subject adjacent to the hyperthermic region.
